(19) United States
(12) Patent Application Publication
(10) Pub. No.: US 2017/0333521 A1
(43) Pub. Date: Nov. 23, 2017

(54) METHOD FOR TARGETED INTRAPROSTATIC ADMINISTRATION OF PRX302 FOR TREATMENT OF PROSTATE CANCER

(71) Applicant: Sophiris Bio Inc., Vancouver (CA)

(72) Inventors: Allison J. HULME, La Jolla, CA (US); Hash AHMED, London (GB); Mark EMBERTON, London (GB)

(21) Appl. No.: 15/418,571

(22) Filed: Jan. 27, 2017

Related U.S. Application Data
(60) Provisional application No. 62/287,873, filed on Jan. 27, 2016.

(51) Int. Cl. A61K 38/17; C07K 14/195; C07K 17/00; C07K 1/00; A61K 38/00

(52) U.S. Cl. CPC A61K 38/17; C07K 14/195; C07K 2319/30; A61K 38/00; C07K 1/00; C07K 2319/33; C07K 17/00

(57) ABSTRACT

This application relates to methods of targeted focal treatment for localized prostate cancer.

This application claims the benefit of U.S. Provisional Application No. 62/287,873 filed Jan. 27, 2016, the disclosure of which is hereby incorporated by reference as if written herein in its entirety.

### FIELD OF THE DISCLOSURE

This application relates to methods for targeted intraprostatic administration of PRX302 for treatment of prostate cancer.

### BACKGROUND OF THE DISCLOSURE

Prostate cancer is the second most common male malignancy. The rising number of men diagnosed with prostate cancer is a result of increasing life expectancy along with the current practice of formal and informal screening using prostate-specific antigen (PSA) blood tests.

However, since the PSA screening era began, there has been a shift in disease profile, with increased detection of low volume and low risk disease. As a result, the risk of over-treatment, and treatment-related harms, is significant. Even the benefits of treating intermediate disease has shown to be equivocal. For example, men with a life expectancy of at least 10 years are currently being offered radical treatments, with the expectation that their life will be prolonged.

Knowledge of which men will benefit from therapy is evolving. It is inherently linked to what burden of cancer is clinically significant and requires treatment, and what threshold of disease can be monitored over time. One of the problems with the current choices men with localised prostate cancer face is that the options for management sit at the extremes of care. At one extreme lies radical treatment, which has significant treatment related morbidity. At present men can expect the following rates of toxicity from radical treatments: 30-90% erectile dysfunction, 5-20% incontinence and 5-20% rectal toxicity. At the other end of the extremes of care lies active surveillance. This has demonstrated excellent medium term survival. However, clinicians and patients have raised concerns over the burden of clinical follow-up, PSA blood tests and biopsy procedures (with their associated risks), and the potential psychological morbidity of living with untreated disease until curative treatment is sought. The complex psychological impact that results from a cancer diagnosis is demonstrated by a significant proportion of men electing to have treatment (about 10% in most series), despite an absence of biochemical or histopathological progression during a period of active surveillance.

The increasing use of multi-parametric magnetic resonance imaging (mpMRI) coupled with targeted biopsies to a lesion means that a more accurate risk stratification is now increasingly possible and men with clinically significant disease are better identified. Clinically significant disease can be defined both by volume or Gleason grade. In the former, it is now well accepted that a Gleason score 6 lesion must be approximately >5 mm maximum cancer core length (MCCL). Any Gleason score 7 or greater is generally accepted as significant regardless of MCCL. Such a paradigm shift is now allowing men to make informed decisions about treatment and active surveillance.

### SUMMARY OF THE DISCLOSURE

Several alternative approaches to the treatment of prostate cancer have been proposed. One has been to develop methods to aggressively screen for local disease while it is still in the prostate and thus potentially treatable by definitive local therapy. Localized cancers are often moderately differentiated and smaller in volume. During the last several decades, there have been improvements to the surgical and radiotherapeutic management of localized prostate cancer.

As disclosed herein are methods for treating prostate cancer in a subject, comprising contacting prostate cancer cells of the subject with a one-time administration of PRX302.

As disclosed herein are methods for treating prostate cancer in a subject, comprising contacting the prostate cancer cells of the subject with one or more doses of PRX302.

The methods as described herein, wherein the one-time administration of PRX302 is directly into and around a pre-identified, clinically significant prostate tumor of a subject.

The methods as described herein, wherein the one-time administration of PRX302 is intratumorally and/or intraprostatically.

The methods as described herein, wherein the clinically significant tumor is a localized prostate tumor.

The methods as described herein, wherein the clinically significant tumor is a metastatic prostate tumor.

The methods as described herein, wherein the one-time administration of PRX302 is up to 5 μg/g prostate.

The methods as described herein, wherein the one-time administration of PRX302 is up to 12 μg/g prostate.

The methods as described herein, wherein the one-time administration of PRX302 is greater than 12 μg/g prostate.

The methods as described herein, wherein the one-time administration of PRX302 is between 200 and 1,000 μg/g tumor.

The methods as described herein, wherein the one-time administration of PRX302 is greater than 1,000 μg/g tumor.

The methods as described herein wherein the concentration of PRX302 ranges from 20 μg/mL to 170 μg/mL.

The methods as described herein wherein the concentration of PRX302 is greater than 170 μg/mL.

The methods as described herein, wherein the one-time administration of PRX302 results in a reduction in prostate tumor volume.

The methods as described herein, wherein the one-time administration of PRX302 results in a reduction of a metastatic prostate tumor.

The methods as described herein, wherein the one-time of PRX302 administration results in treatment of the metastatic prostate tumor.

The methods as described herein wherein the one-time of PRX302 administration results in a reduction in Gleason pattern.

The methods as described herein, wherein the one-time of PRX302 administration results in a reduction in maximum cancer core length or Gleason pattern.

The methods as described herein, wherein one-time of PRX302 administration results complete tumor ablation or down-grade from clinically significant to non-significant, which can be described as a reduction of MCCL or Gleason grade.

The methods as described herein, wherein tumor ablation is confirmed on re-biopsy.

The methods as described herein, wherein tumor ablation is confirmed on mpMRI.

### BRIEF DESCRIPTION OF THE FIGURES

FIG. 1A. Represents a lesion with volume ≧0.5 mL and presence of dominant Gleason pattern 4.

FIG. 1B. Represents lesions with volume ≧0.2 mL lesions and any Gleason pattern 4.

FIG. 1C. Consistent with very low risk cancers (and possibly indolent lesions of epithelial origin, or ‘IDLE’, lesions). TCCL: Total Cancer Core Length. MCCL: Maximum Cancer Core Length.

### DETAILED DESCRIPTION OF THE EMBODIMENTS OF THE DISCLOSURE

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise.

Unless explained otherwise, all technical and scientific terms used herein have the same meaning as commonly understood to one of ordinary skill in the art to which this disclosure belongs.

In one embodiment, enhance, is to improve the quality, amount, or strength of something. In one embodiment, a therapy enhances the ability of a subject to reduce tumors, such as a prostate carcinoma, in the subject if the subject is more effective at fighting tumors. In another embodiment, a therapy enhances the ability of an agent to reduce tumors, such as a prostate carcinoma, in a subject if the agent is more effective at reducing tumors. Such enhancement can be measured using the methods disclosed herein, for example determining the decrease in tumor volume.

A therapeutically effective amount is an amount sufficient to achieve a desired biological effect, for example an amount that is effective to decrease the size (i.e. volume), severity/clinical significance/Gleason grade, side effects and/or metastasis of prostate cancer. In one example, it is an amount sufficient to decrease the symptoms or effects of a prostate carcinoma, such as the size of the tumor. In particular examples, it is an amount effective to decrease the size of a prostate tumor and/or prostate metastasis by at least 30%, 40%, 50%, 70%, 80%, 90%, 95%, 99% or even 100% (complete elimination of the tumor).

In particular embodiments, it is an amount of PRX302 (topsalysin) effective to decrease a prostate tumor and/or an amount of prostate cancer cells lysed by PRX302, such as in a subject to whom it is administered, for example a subject having one or more prostate carcinomas. In other embodiments, it is an amount of PRX302, and/or an amount of prostate cancer cells lysed by PRX302, effective to decrease the risk of metastasis of a prostate carcinoma—i.e. by reducing the malignant Gleason grade.

In one embodiment, the therapeutically effective amount also includes a quantity of PRX302 and/or an amount of prostate cancer cells lysed by PRX302 sufficient to achieve a desired effect in a subject being treated. For instance, these can be an amount necessary to improve signs and/or symptoms a disease such as cancer, for example prostate cancer.

In another embodiment, an effective amount of PRX302 and/or prostate cancer cells lysed by PRX302 can be administered in a single dose, or in several doses, for example daily, during a course of treatment. However, the effective amount of will be dependent on the subject being treated, the severity and type of the condition being treated, and the manner of administration. For example, in one embodiment, a therapeutically effective amount of PRX302 can be administered to prostates weighing at least 20 g and having tumors in size of 0.1-0.8 g, the total dose administered was up to 5 μg/g prostate up to 1,000 μg/g tumor. In another embodiment, the therapeutically effective amount of PRX302 will be between 200 and 1,000 μg/g tumor, depending on the size of the tumor, the total prostate volume (PV), and an upper dose limit of 12 μg/g prostate. In yet another embodiment, the therapeutically effective amount of PRX302 is greater than 1,000 μg/g tumor.

A therapeutically effective dose, in one example, is a dose of PRX302, sufficient to decrease tumor cell volume, such as a prostate carcinoma, in a subject to whom it is administered, resulting in a regression of a pathological condition, or which is capable of relieving signs or symptoms caused by the condition. In a particular example, it is a dose of PRX302 sufficient to decrease metastasis of a prostate cancer.

In yet another example, it is a dose of cell lysate resulting from contact of cells with PRX302 sufficient to decrease tumor cell volume, such as a prostate carcinoma, in a subject to whom it is administered, resulting in a regression of a pathological condition, or which is capable of relieving signs or symptoms caused by the condition. In a particular example, it is a dose of cell lysate resulting from contact of cells with PRX302 sufficient to decrease metastasis of a prostate cancer.

A tumor is a neoplasm. This includes but is not limited to solid tumors.

Examples of solid tumors, such as sarcomas and carcinomas, include, but are not limited to fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, kidney cancer, thyroid cancer, colorectal cancer, bladder cancer, stomach cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Disclosure of certain specific examples is not meant to exclude other embodiments. In addition, any treatments described herein are not necessarily exclusive of other treatment, but can be combined with other bioactive agents or treatment modalities.

### Rationale

A selective targeted tissue-preserving approach for treatment of prostate cancer will allow the treatment to conform more closely to the area(s) of cancer, with preservation of surrounding normal prostatic tissue. This concept has been termed ‘focal therapy’, and encompasses a range of therapeutic protocols that offer a tissue-sparing approach with the aim of reducing the treatment insult to the surrounding anatomical structures, and consequently, potentially leading to lower rates of genitourinary side-effects whilst retaining the cancer control benefits that whole-gland therapies offer. There has been growing interest in the potential role of focal therapy as a treatment for localized prostate cancer.

Such a proposed change in treatment of prostate cancer reflects the management of almost all other solid organ cancers, in which organ preservation is fundamental to functional preservation (breast, kidney, liver, pancreas, thyroid). It is also carried out in other hollow organ cancers (colorectal, bladder, stomach, lung). To achieve selective treatment of the prostate gland, technologies are required that can localize clinically significant areas of prostate cancer precisely. Multi-parametric MRI and transperineal and/or transrectal biopsies (targeted and/or mapping) closely match the optimal attributes for this requirement. Technologies are also needed that can treat discrete areas of tissue. Several ablative therapies are already available within clinical practice and research, including HIFU, cryosurgery, photodynamic therapy, brachytherapy and radiofrequency ablation, and thermal lasers.

All therapies are at different stages of development in relation to their use as technologies capable of focal ablation. More recently, studies have been started for evaluation of radiotherapy techniques, e.g., low dose rate radioactive seed brachytherapy, in the focal treatment setting. Early published study data have demonstrated promising results for obtaining early oncological outcome together with preservation of genitourinary function. On average, studies in cryosurgery and HIFU focal ablation have shown incontinence rates below 5% and impotence rates of 5-10%.

### Disease Detection and Localization

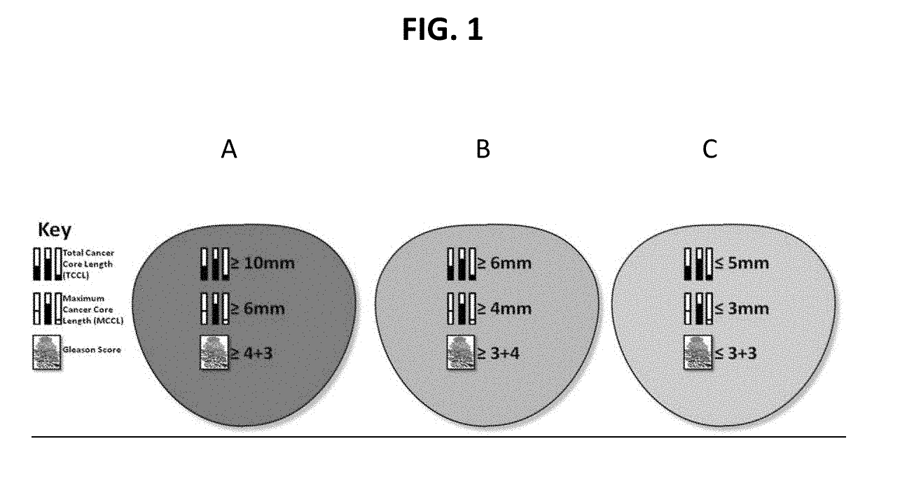

Effective, selective, targeted focal ablation relies on accurate localisation and targeting of disease, minimizing treatment to surrounding normal tissue. The biopsy and imaging techniques are mpMRI and targeted transperineal/transrectal biopsies. A classification system validated on radical prostatectomy specimens was developed using computer simulation work for transperineal template biopsies (see FIG. 1). Although this represents the ideal setting for template biopsies, this classification system all defines what minimum amount of cancer within positive biopsy cores accurately represents a significant and insignificant lesion.

In men with Gleason pattern 3+3 we have stipulated a lower limit of maximum cancer core length that must be exceeded to fulfil inclusion criteria. The lower limit for inclusion has been set at 5 mm maximum cancer core length.

Men with Gleason pattern 7 (3+4 or 4+3) are considered to have clinically significant disease.

### PRX302: Structure and Mechanism of Action

PRX302 (topsalysin), a novel, first-in-class, investigational pore-forming protein is currently under development for the treatment of lower urinary tract symptoms (LUTS) in men with moderate to severe benign prostatic hyperplasia (BPH) and for the treatment of men with prostate cancer. PRX302 is administered via direct intraprostatic injection and does not require complicated equipment or substantial additional clinician training, in part due to the similarity to routine prostate biopsy.

PRX302 (53 kD, 476 amino acids) is a genetically-engineered recombinant version of a native bacterial pore-forming protein (proaerolysin) using an Aeromonas salmonicida expression system. The native furin recognition and activation sequence for conversion of pro-aerolysin to active aerolysin has been replaced with a peptide sequence that is recognised and activated only by PSA. PSA is a serine protease produced by epithelial prostate cells, and is active in the prostate, normal seminal fluid, and the extracellular fluid surrounding prostate cancer cells, but any PSA that leaks into the blood circulation is inactivated through the formation of covalent complexes with abundant serum protease inhibitors.

Activation of PRX302 by PSA occurs following PRX302 rapid binding to glycophosphatidyl-inositol (GPI)-anchored proteins abundantly expressed on the surface of prostate cells. Release of the C-terminal inhibitory peptide of PRX302 by PSA cleavage results in a conformational change in the aerolysin protein, causing oligomerisation and rapid and irreversible insertion into the plasma membrane. The resulting aerolysin heptamer pore spans the cell membrane resulting in ion leakage, cell swelling, rapid loss of membrane integrity, and subsequent cell death.

### EXAMPLES

### Example 1

### Identifying the Disease by Multi-Parametric MRI and Transperineal Biopsy

Multi-parametric MRI (mpMRI) will be the non-invasive investigation on which the presence of a histologically proven, clinically significant lesion amenable to focal ablation will be identified. This will already have been performed, prior to invitation to participate in the described. However, if the mpMRI was obtained greater than 6 months prior to the planned dosing in this study, an additional mpMRI will be obtained at screening.

Pre-treatment and all post-treatment imaging will be performed using either a 1.5 Tesla or 3 Tesla scanner. Men were scanned on the same magnetic field strength throughout the study. A full protocol of T1 and T2 weighted turbo-spin echo images and a dynamic post gadolinium volume acquisition will be used for both pre-treatment diagnostic and planning scans and post-treatment assessment of the effect of PRX302.

### Example 2

### Disease Localization: Transperineal Prostate Biopsies

The initial transperineal biopsy will already have been performed, prior to invitation to participate in the study, and will demonstrate eligibility for inclusion in this study. The transperineal or transrectal targeted biopsy will need to be concordant with the lesion seen on the mpMRI.

Image registration will be used to fuse the mpMRI images to the ultrasound images during the injection of PRX302 in order to more accurately facilitate targeting of the lesion by injection based on the imaging phenotype.

### Example 3

### Study Drug Treatment of Patients

PRX302 (topsalysin) is an investigational, genetically-modified, pore-forming protein with the native furin protease activation site of the proaerolysin molecule replaced by an amino acid sequence that is highly specific to only enzymatically-active PSA. PRX302 remains inactive in the absence of enzymatically-active PSA and is not activated by PSA remote from prostate cells, such as PSA in the systemic circulation. After activation, PRX302 spontaneously oligomerises into heptamers that insert irreversibly through the cell membrane, leading to cell death.

The study drug was supplied as a single-use, 2 mL vial with 0.5 mL fill, frozen (−20±5° C.), consisting of PRX302 drug product in aqueous solution at a concentration of 300 μg/mL.

Diluent for study drug preparation is recombinant human serum albumin (rHSA) 2% weight/volume (w/v) (0.02 g/mL) in phosphate buffered saline provided as 20 mL in a 20 mL vial refrigerated (5±3° C.).

### Example 4

### Study Design I

Eighteen men with histologically proven, clinically significant, localized, low to intermediate-risk prostate cancer were selected. The men all are aged at least 40 years old and have a life expectancy of at least 10 years. Serum PSA levels are equal to or less than 15 ng/mL. Patients have a clinically significant tumor/visible lesion on mpMRI that is accessible to PRX302 transperineal injection. Patients have histologically proven prostate cancer with a maximum Gleason score of 7. If the Gleason total score is 6, the MCCL must exceed 5 mm.

For previously obtained patient mpMRI images were mapped to real time 3D ultrasound images using fusion software to facilitate the injection of PRX302 into a pre-identified, histologically proven, clinically significant lesion.

The eligible men who were selected for the study had a single lesion injected transperineally, under general anaesthetic with up to 5 mL of a 20 ug/mL PRX302 dosing solution (the maximum does of 5 ug/g prostate)

### Example 5

### Study Drug Dosing

Up to a total of 5 mL (maximum of 300 μg) of the prepared study drug dosing solution (PRX302 at a fixed concentration of 20 μg/mL) will be injected transperineally into the pre-identified lesion in aliquots of 1 mL. A 5 mm template will be used to guide each 1 mL injection up to a maximum of 5 injections into and around the pre-identified lesion.

See Table 1 for the amount of drug injected based upon different prostate volumes.

TABLE 1
Amount of Study Drug (μg/g of Prostate) Injected
Based upon Different Prostate Volumes.
Prostate Volume (g)

| Volume of PRX302 dosing solution | | | | | |
|---|---|---|---|---|---|
| (20 ug/mL) Injected | 20 | 30 | 50 | 80 | 100 |
| 1 | 1.00 | 0.67 | 0.40 | 0.25 | 0.20 |
| 2 | 2.00 | 1.33 | 0.80 | 0.50 | 0.40 |
| 3 | 3.00 | 2.00 | 1.20 | 0.75 | 0.60 |
| 4 | 4.00 | 2.67 | 1.60 | 1.00 | 0.80 |
| 5 | 5.00 | 3.33 | 2.00 | 1.25 | 1.00 |

### Example 6

### Results from Study Design I

In prostates weighing at least 20 g and having tumors in size of 0.1-0.8 g, the total dose administered was up to 5 μg/g prostate and up to 1,000 m/g tumor. Notably in the 3 patients who positively responded to PRX302, defined as having a lesion that was no longer clinically significant at 24 weeks after treatment, their dose normalized to tumor size was 500-1,000 m/g tumor. Nine of the patients were non-responders (i.e., no change or a slow progression of their disease), and they had received PRX302 doses of typically less than 500 m/g tumor. The remaining 6 patients were deemed partial responders (e.g., improvement in Gleason pattern, or reduction in MCCL) and they received PRX302 doses spanning the entire range up to 1,000 m/g tumor. Thus, the data suggest that a higher local dose to the tumor may be one factor that increases potential lesion ablation, leading to a more favorable clinical outcome.

TABLE 2
Baseline Demographics.

| | | Standard | | |
|---|---|---|---|---|
| | Mean | Deviation | Median | IQR |
| Tumor Size (cm³) | 0.4 | 0.5 | 0.3 | 0.3 |
| PSA Level (ng/ml) | 6 | 1.9 | 6.3 | 2.5 |

TABLE 3
Gleason Score.

| | Patients at Baseline (N) | Patients at Baseline (%) |
|---|---|---|
| Gleason 6 (3 + 3) | 4 out of 18 | 22% |
| Gleason 7 (4 + 3) | 2 out of 18 | 11% |
| Gleason 7 (3 + 4) | 12 out of 18 | 67% |

Six month biopsy data was obtained on all 18 patients treated. Two men experienced complete ablation of their target tumors. Seven men experienced a partial response to treatment either a reduction in the maximum core length or a reduction in Gleason pattern. Nine men had no response to treatment.

TABLE 4
Six Month Biopsy Data.

| | | Lesion Size (ml); | | Gleason |
|---|---|---|---|---|
| | PV | Dose (ug/tumor); | MCCL(mm) | Score |
| Pt | (g) | No Injections | Pre −> Post | Pre −> Post |
| R | 47 | 0.1; (1000); 5 | 6.0 −> 0.0 | 3 + 4 −> 0 |
| R | 46 | 0.2; (500); 5 | 5.0 −> 0.0 | 3 + 4 −> 0 |
| R | 35 | 0.2; (600); 6 | 7.0 −> 3.0 | 3 + 3 −> 3 + 3 |
| P | 21 | 0.5; (164); 6 | 2.0 −> 7.0 | 3 + 4 −> 3 + 3 |
| P | 27 | 0.3; (333); 5 | 6.0 −> 4.0 | 3 + 4 −> 3 + 3 |
| P | 71 | 0.2; (500); 5 | 4.0 −> 6.0 | 3 + 4 −> 3 + 3 |
| P | 40 | 0.7; (143); 5 | 2.0 −> 2.0 | 4 + 3 −> 3 + 4 |
| P | 32 | 0.2; (500); 5 | 7.0 −> 5.0 | 3 + 4 −> 3 + 4 |
| P | 33 | 0.1; (1000); 7 | 4.0 −> 3.0 | 3 + 4 −> 3 + 4 |
| N | 20 | 0.8; (101); 4 | 7.0 −> 4.0 | 3 + 4 −> 4 + 3 |
| N | 60 | 0.5; (200); 6 | 6.0 −> 5.5 | 3 + 3 −> 3 + 3 |
| N | 31 | 0.5; (200); 6 | 2.0 −> 9.0 | 4 + 3 −> 4 + 3 |
| N | 34 | 0.4; (250); 5 | 4.0 −> 5.0 | 3 + 4 −> 3 + 4 |
| N | 74 | 0.1; (1000); 6 | 2.0 −> 5.0 | 3 + 4 −> 3 + 4 |
| N | 31 | 0.7; (143); 7 | 5.0 −> 11.0 | 3 + 4 −> 3 + 4 |
| N | 40 | 0.2; (500); 5 | 5.0 −> 9.0 | 3 + 3 −> 3 + 4 |
| N | 99 | 2.2; (45); 5 | 9.0 −> 10.0 | 3 + 3 −> 3 + 5 |

R: Responder
P: Partial Responder
N: Non-Responder

### Example 7

### Study Design II

PRX302 doses in Study Design II will be between 200 and 1,000 μg/g tumor depending on the size of the tumor, the total prostate volume (PV) and an upper dose limit of 12 μg/g prostate. Support for this dose range comes from Study Design I (Examples 4-6 with targeted intraprostatic injection of PRX302 in 18 men with histologically proven, clinically significant, localised, low- to intermediate-risk prostate cancer.

The desired PRX302 dose range of 200-1,000 μg/g tumor will be delivered in a volume of 6 mL for total PVs of 20 g and higher. Thus, the maximum volume injected into the prostate will be ≦30%. This amount of drug product has been administered with no observed clinical sequelae due to the amount of volume delivered in previous clinical studies with PRX302 in prostate cancer and BPH (enlarged prostates are more susceptible to volume load and negative effects on the lower urinary tract). The dose will be delivered in aliquots no smaller than 1 mL, via guided transrectal ultrasound (TRUS) into and around the pre-identified target lesion, which is intended to amplify the pharmacodynamic effects of PRX302 directly on the tumor cells.

Unlike Study Design I, PRX302 in Study Design II will not be manually injected but rather once the Investigator has placed the needles into and around the pre-identified tumor for treatment, the needle will be attached to either an infusion pump or a springfusor in order to allow the study drug to slowly diffuse from the needle tip. This delivery of PRX302 is intended to minimize the setting up of “microjects” allowing the drug to be deflected away from the intended site of injection by the densely-packed cells of the tumor.

Study Design II also includes an option to potentially re-treat the targeted lesion 26 weeks after the first PRX302 dose with a second dose of PRX302 in patients who qualify based on safety and evidence of pharmacological activity of PRX302 and some clinical effect. Specifically, patients eligible in the opinion of the Investigator to receive a second dose will need to have no clinically significant adverse effects attributable to study drug or the dosing procedure, a clinical response to the first PRX302 dose and the persistent presence of a clinically significant tumor. Therefore, patients with complete ablation of their tumor will not be retreated. The determination of a clinical response will take into account not only changes in tumor size, but also changes in the Gleason score. Successful cumulative damage to prostate tissue from a second intraprostatic dose of PRX302 has been shown in monkeys in which the two doses were equal (˜14 μg/g prostate) and administered 8 weeks apart. Both doses were systemically well tolerated, and there was recovery of the prostate at the end of each dosing period. In this study protocol, there is an added safety measure by having more time for prostate recovery with the doses spaced 26 weeks apart.

In Study Design II, the doses selected will provide data to guide the selection of the optimal efficacious dose of PRX302 for pivotal studies in the indication of the treatment for low- to intermediate-risk prostate cancer while balancing the safety of subjects. The potential benefit versus the potential risk to patients enrolled in this study is considered favorable such that an evaluation of the efficacy and safety of PRX302 as outlined in the study protocol is justified for a patient population with few and often unsatisfactory treatment options.

We claim:

1. A method for treating or reducing the size of prostate cancer in a subject in need thereof, comprising delivering a one-time dose of PRX302 directly into or around a pre-identified, clinically significant lesion guided by high-intensity focused ultrasound.

2. A method for treating or reducing the size of prostate cancer in a subject in need thereof, comprising delivering one or more doses of PRX302 directly into or around a pre-identified clinically significant lesion guided by high-intensity focused ultrasound.

3. The method of claim 1, wherein the one-time dose of PRX302 is administered intratumorally and/or intraprostatically.

4. The method of claim 1, wherein the one-time dose of PRX302 will be between 200 and 1,000 μg/g tumor.

5. The method of claim 1, wherein the one-time dose of PRX302 is greater than 1,000 μg/g tumor.

6. The method of claim 1, wherein the one-time dose of PRX302 is ranges from 20 μg/mL to 170 μg/mL.

7. The method of claim 1, wherein the one-time dose of PRX302 is greater than 170 μg/mL.

8. The method of claim 1, wherein the one-time dose of PRX302 is delivered in aliquots not smaller than 1 mL.

9. The method of claim 1, wherein the one-time dose wherein the one-time dose of PRX302 is up to 5 μg/g prostate.

10. The method of claim 1, wherein the one-time dose wherein the one-time dose of PRX302 is up to 12 μg/g prostate.

11. The method of claim 1, wherein the one-time dose wherein the one-time dose of PRX302 is greater than 12 μg/g prostate.

12. The method of claim 1, wherein the clinically significant lesion is a localized prostate tumor.

13. The method of claim 1, wherein the clinically significant lesion is a metastatic prostate tumor.

14. The method of claim 1, wherein the one-time dose of PRX302 results in a reduction in prostate tumor volume.

15. The method of claim 1, wherein the one-time administration of PRX302 results in a reduction of a metastatic prostate tumor.

16. The method of claim 1, wherein the one-time of PRX302 administration results in treatment of the metastatic prostate tumor.

17. The method of claim 1, wherein the one-time of PRX302 administration results in a reduction in Gleason pattern.

18. The method of claim 1, wherein the one-time of PRX302 administration results in a reduction in maximum cancer core length or Gleason pattern.

19. The method of claim 1, wherein the high-intensity focused ultrasound is guided transrectally.

20. The method of claim 1, wherein the one-time of PRX302 administration results complete tumor ablation.

21. The method of claim 20, wherein tumor ablation is confirmed on re-biopsy.

22. The method of claim 20, wherein tumor ablation is confirmed on mpMRI.
